(19) United States
(12) Patent Application Publication
(10) Pub. No.: US 2023/0021242 A1
(43) Pub. Date: Jan. 19, 2023

(54) METHOD OF HYDROLYSIS OF DIMETHYL SUCCINYL SUCCINATE

(71) Applicant: Xi'an Sunward Aeromat Co., Ltd., Xi'an (CN)

(72) Inventors: Longlong XU, Xi'an (CN); Junmiao TAO, Xi'an (CN); Shaodan ZHAO, Xi'an (CN); Bing WEI, Xi'an (CN); Zhenguo ZHANG, Xi'an (CN); Wenyou WANG, Xi'an (CN); Hui KANG, Xi'an (CN); Gang WANG, Xi'an (CN)

(21) Appl. No.: 17/948,203

(22) Filed: Sep. 19, 2022

Related U.S. Application Data
(63) Continuation-in-part of application No. PCT/CN2021/128703, filed on Nov. 4, 2021.

(30) Foreign Application Priority Data
Dec. 24, 2020 (CN) 202011552754.X

(51) Int. Cl. C07C 69/40; B01J 23/72; B01J 23/755; B01J 23/80; B01J 23/84; B01J 31/02

(52) U.S. Cl. CPC C07C 69/40; B01J 23/72; B01J 23/755; B01J 23/80; B01J 23/84; B01J 31/0225; B01J 31/0232

(57) ABSTRACT

A method of hydrolysis of dimethyl succinyl succinate includes: adding DMSS and water to a reactor, and stirring; adding a phase transfer catalyst to the reactor, and heating; and adding an acid and a transition metal salt to the reactor for hydrolysis of DMSS. The acid is sulfuric acid, hydrochloric acid or nitric acid, and the W ion concentration of the mixture in the reactor is 0.2-12 mol/L. The transition metal salt is a nitrate, sulfate, or chloride of copper, nickel, zinc or manganese, or a combination thereof; and the metal ion concentration of the mixture in the reactor is 0.01-0.1 mol/L.

### CROSS-REFERENCE TO RELATED APPLICATIONS

This application is a continuation-in-part of International Patent Application No. PCT/CN2021/128703 with an international filing date of Nov. 4, 2021, designating the United States, now pending, and further claims foreign priority benefits to Chinese Patent Application No. 202011552754.X filed Dec. 24, 2020. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass, 02142.

### BACKGROUND

The disclosure relates to the field of organic chemical industry, and more particularly to a method of hydrolysis of dimethyl succinyl succinate.

1,4-cyclohexanedione, having a molecular formula of C₆H₈O₂, is a chemical intermediate, which is widely used to produce drugs, pesticides, liquid crystal materials, organic photoelectric materials, superconducting materials, etc.

Typically, 1,4-cyclohexanedione is produced by hydrolysis of dimethyl succinyl succinate (DMSS). Conventional methods for hydrolysis of DMSS are time-consuming, and produce byproducts that are difficult to remove.

### SUMMARY

One objective of the disclosure is to provide a method of hydrolysis of dimethyl succinyl succinate.

The disclosure provides a method of hydrolysis of dimethyl succinyl succinate (DMSS), the method comprising:

1) adding DMSS and water to a reactor, and stirring;

2) adding a phase transfer catalyst to the reactor, and heating; and

3) adding an acid and a transition metal salt to the reactor for hydrolysis of DMSS;

the acid is sulfuric acid, hydrochloric acid or nitric acid, and a H⁺ ion concentration of a mixture in the reactor is 0.2-12 mol/L; and

the transition metal salt is a nitrate, sulfate, or chloride of copper, nickel, zinc or manganese, or a combination thereof; and a metal ion concentration of the mixture in the reactor is 0.01-0.1 mol/L.

In a class of this embodiment, in 1), an addition amount of the water is 5-20 times that of DMSS by weight.

In a class of this embodiment, in 2), the phase transfer catalyst is dodecyl benzene sulfonic acid, sodium dodecyl benzene sulfonate, polyethylene glycol, dodecyl trimethyl ammonium chloride or tetrabutyl ammonium bromide.

In a class of this embodiment, an addition amount of the phase transfer catalyst accounts for 0.01-0.1 wt. % that of the water.

In a class of this embodiment, the mixture in the reactor is heated to 60-100° C., and then the acid and the transition metal salt are added to the reactor.

The following advantages are associated with the preparation method of the disclosure:

1. The method is easy to operate, and the entire reaction time is about 4-60 hours, which is much shorter than conventional hydrolysis time.

2. The conversion rate of DMSS is up to 100%, and the selectivity of the product 1,4-cyclohexanedione is high, up to more than 98%, which is due to the coordination between metal cations and the six membered ring, which increases the stability of the six membered ring.

3. No organic solvent is involved in the hydrolysis reaction. With the addition of the phases transfer catalyst, the contact area between DMSS and water is increased, which saves the use of the organic solvent and reduces the amount of waste liquid.

### DETAILED DESCRIPTION

To further illustrate, embodiments detailing a method of hydrolysis of dimethyl succinyl succinate are described below. It should be noted that the following embodiments are intended to describe and not to limit the disclosure.

A method of hydrolysis of dimethyl succinyl succinate (DMSS) comprises:

1. adding DMSS and water to a reactor, and stirring, where the addition amount of the water is 5-20 times that of DMSS by weight;

2. adding a phase transfer catalyst to the reactor, and heating; and

3. adding an acid and a transition metal salt to the reactor for hydrolysis of DMSS when the mixture in the reactor is heated to 60-100° C.

The phase transfer catalyst is dodecyl benzene sulfonic acid, sodium dodecyl benzene sulfonate, polyethylene glycol, dodecyl trimethyl ammonium chloride or tetrabutyl ammonium bromide. The addition amount of the phase transfer catalyst accounts for 0.01-0.1 wt. % that of the water.

The acid is sulfuric acid, hydrochloric acid or nitric acid, and a H⁺ ion concentration of a mixture in the reactor is 0.2-12 mol/L.

The transition metal salt is a nitrate, sulfate, or chloride of copper, nickel, zinc or manganese, or a combination thereof; and the metal ion concentration of the mixture in the reactor is 0.01-0.1 mol/L.

### Example 1

200 g of DMSS and 1 L of water were mixed and stirred. 0.1 g of dodecyl benzene sulfonic acid was added to the mixture, and heated to 60° C. Thereafter, 10 g of concentrated sulfuric acid and 2.4 g of copper nitrate trihydrate were added to the mixture. 60 hours later, an orange clear solution was obtained. Chromatographic analysis showed, the conversion rate of DMSs was 100% and the selectivity of 1,4-cyclohexanedione was 90.5%.

### Example 2

200 g of DMSS and 1 L of water were mixed and stirred. 0.3 g of sodium dodecyl benzene sulfonate was added to the mixture, and heated to 70° C. Thereafter, 25 g of concentrated sulfuric acid and 6.8 g of zinc chloride were added to the mixture. 50 hours later, a faint yellow clear solution was obtained. Chromatographic analysis showed, the conversion rate of DMSs was 100% and the selectivity of 1,4-cyclohexanedione was 95.1%.

### Example 3

200 g of DMSS and 1 L of water were mixed and stirred. 1.0 g of polyethylene glycol was added to the mixture, and heated to 80° C. Thereafter, 156 g of concentrated sulfuric acid and 26.3 g of nickel sulfate hexahydrate were added to the mixture. 35 hours later, a faint yellow clear solution was obtained. Chromatographic analysis showed, the conversion rate of DMSs was 100% and the selectivity of 1,4-cyclohexanedione was 98.3%.

### Example 4

200 g of DMSS and 1 L of water were mixed and stirred. 0.5 g of dodecyl benzene sulfonic acid was added to the mixture, and heated to 100° C. Thereafter, 792 g of concentrated sulfuric acid and 23.9 g of 50% manganese nitrate aqueous solution were added to the mixture. 4 hours later, a brown clear solution was obtained. Chromatographic analysis showed, the conversion rate of DMSs was 100% and the selectivity of 1,4-cyclohexanedione was 90.2%.

### Example 5

200 g of DMSS and 2 L of water were mixed and stirred. 1 g of dodecyl trimethyl ammonium chloride was added to the mixture, and heated to 80° C. Thereafter, 503 g of concentrated sulfuric acid and 32.7 g of zinc nitrate hexahydrate were added to the mixture. 24 hours later, a faint yellow clear solution was obtained. Chromatographic analysis showed, the conversion rate of DMSs was 100% and the selectivity of 1,4-cyclohexanedione was 98.5%.

### Example 6

200 g of DMSS and 4 L of water were mixed and stirred. 2 g of dodecyl trimethyl ammonium chloride was added to the mixture, and heated to 80° C. Thereafter, 1000 g of concentrated sulfuric acid and 55.6 g of zinc nitrate hexahydrate were added to the mixture. 18 hours later, a faint yellow clear solution was obtained. Chromatographic analysis showed, the conversion rate of DMSs was 100% and the selectivity of 1,4-cyclohexanedione was 98.0%.

### Example 7

200 g of DMSS and 2 L of water were mixed and stirred. 1 g of tetrabutyl ammonium bromide was added to the mixture, and heated to 80° C. Thereafter, 793 g of concentrated hydrochloric acid and 39.6 g of zinc nitrate hexahydrate were added to the mixture. 40 hours later, a faint yellow clear solution was obtained. Chromatographic analysis showed, the conversion rate of DMSs was 100% and the selectivity of 1,4-cyclohexanedione was 94.5%.

### Example 8

200 g of DMSS and 2 L of water were mixed and stirred. 1 g of dodecyl trimethyl ammonium chloride was added to the mixture, and heated to 80° C. Thereafter, 703 g of concentrated nitric acid and 36.5 g of zinc nitrate hexahydrate were added to the mixture. 40 hours later, a faint yellow clear solution was obtained. Chromatographic analysis showed, the conversion rate of DMSs was 100% and the selectivity of 1,4-cyclohexanedione was 94.6%.

It will be obvious to those skilled in the art that changes and modifications may be made, and therefore, the aim in the appended claims is to cover all such changes and modifications.

What is claimed is:

1. A method of hydrolysis of dimethyl succinyl succinate (DMSS), the method comprising:
1) adding DMSS and water to a reactor, and stirring;
2) adding a phase transfer catalyst to the reactor, and heating; and
3) adding an acid and a transition metal salt to the reactor for hydrolysis of DMSS;
wherein
the acid is sulfuric acid, hydrochloric acid or nitric acid, and a H⁺ ion concentration of a mixture in the reactor is 0.2-12 mol/L; and
the transition metal salt is a nitrate, sulfate, or chloride of copper, nickel, zinc or manganese, or a combination thereof; and a metal ion concentration of the mixture in the reactor is 0.01-0.1 mol/L.

2. The method of claim 1, wherein in 1), an addition amount of the water is 5-20 times that of DMSS by weight.

3. The method of claim 1, wherein in 2), the phase transfer catalyst is dodecyl benzene sulfonic acid, sodium dodecyl benzene sulfonate, polyethylene glycol, dodecyl trimethyl ammonium chloride or tetrabutyl ammonium bromide.

4. The method of claim 1, wherein an addition amount of the phase transfer catalyst accounts for 0.01-0.1 wt. % that of the water.

5. The method of claim 1, wherein the mixture in the reactor is heated to 60-100° C., and then the acid and the transition metal salt are added to the reactor.
